(19) United States
(12) Patent Application Publication
(10) Pub. No.: US 2004/0048254 A1
(43) Pub. Date: Mar. 11, 2004

(54) Diagnosis of diseases associated with tumor supressor genes and oncogenes

(76) Inventors: Alexander Olek, Berlin (DE); Kurt Berlin, Stahnsdorf (DE)

Correspondence Address: DAVIDSON, DAVIDSON & KAPPEL, LLC, 485 SEVENTH AVENUE, 14TH FLOOR, NEW YORK, NY, 10018, US

(21) Appl. No.: 10/221,714

(22) PCT Filed: Mar. 15, 2001
(86) PCT No.: PCT/EP01/02955

(30) Foreign Application Priority Data
Mar. 15, 2000 (DE) 100-13-847.0
Apr. 6, 2000 (DE) 100-19-058.8
Apr. 7, 2000 (DE) 100-19-173.8
Jun. 30, 2000 (DE) 100-32-529.7
Sep. 1, 2000 (DE) 100-43-826.1

(51) Int. Cl. C12Q 1/68; C07H 21/04; C12N 9/99; C12P 21/02; C12N 5/06

(52) U.S. Cl. 435/6; 435/69.2; 435/184; 435/320.1; 435/325; 536/23.2

(57) ABSTRACT

The present invention relates to the chemically modified genomic sequences of genes associated with tumor suppressor genes and oncogenes, to oligonucleotides and/or PNA-oligomers for detecting the cytosine methylation state of tumor suppressor genes and oncogenes which are directed against the sequence, as well as to a method for ascertaining genetic and/or epigenetic parameters of tumor suppressor genes and oncogenes.

### FIELD OF THE INVENTION

The levels of observation that have been well studied by the methodological developments of recent years in molecular biology, are the genes themselves, the translation of these genes into RNA, and the resulting proteins. The question of which gene is switched on at which point in the course of the development of an individual, and how the activation and inhibition of specific genes in specific cells and tissues are controlled is correlatable to the degree and character of the methylation of the genes or of the genome. In this respect, pathogenic conditions may manifest themselves in a changed methylation pattern of individual genes or of the genome.

The present invention relates to nucleic acids, oligonucleotides, PNA-oligomers and to a method for the diagnosis and/or therapy of diseases which have a connection with the genetic and/or epigenetic parameters of genes associated with tumor suppressor genes and oncogenes and, in particular, with the methylation status thereof.

### PRIOR ART

There exists a need for the development of alternative methods of treatment and diagnosis of cancer patients. Current methods of treatment, such as radiotherapy and chemotherapy are often accompanied by unpleasant side effects, and are ineffective against many forms of cancers.

The genetics of cancer is complicated, involving multiple dominant, positive regulators of the transformed state (oncogenes) as well as multiple recessive, negative regulators (tumor suppressor genes). About 100 oncogenes have been identified. The oncogenes fall into several groups, representing different types of activities ranging from transmembrane proteins to transcription factors. About 10 tumor suppressor genes are known at present. They represent loss of function in genes that usually impose some constraint on tumor suppressor genes and oncogenes or cell growth; the release of the constraint is tumorigenic. In addition to loss of heterozygosity and spontaneous mutation in genes, there exists a further mechanism which may lead to tumor formation. The epigenetic misregulation of genes has been shown to be involved in the formation of tumors. The best characterised epigenetic parameter is that of genomic methylation, the covalent modification of the C5 position of cytosine. Methylation anomalies in cancer include point mutations, global hypomethylation, hypomethylation of individual genes, hypermethylation of CpG islands and loss of imprinting.

The high rate of mutation within CpG islands can lead to cancer. Methylated cytosines bases may spontaneously deaminate to form uracil. The mismatch repair may then be impaired by DNA-MTase, leading to a cytosine to thiamidine transition. A good example of this is the p53 gene. A mutation in this gene is present in more than 50% of all tumors, of which an estimated 24% are cytosine to thymidine transitions at CpG dinucleotides.

Decreased levels of global methylation is a common in many forms of tumors. Hypomethylation of individual genes has also been observed. An inverse relationship between levels of methylation and cell proliferation has been observed in the bcl-2 gene (lymphocytic leukemia) and the k-ras proto-oncogene in lung and colon carcinomas.

The p16 gene is a key tumor suppressor genes and oncogenes regulatory gene. The p16 protein halts cell-cycle progression at the G1/S boundary, and the loss of p16 function may lead to cancer progression by allowing unregulated cellular proliferation. Hypermethylation mediated inactivation of the p16 gene has been demonstrated in brain, breast, colon, head and neck, and non-small-cell lung cancer and in high grade non-Hodgkin's lymphoma.

The role of methylation in tumorigenesis is reveiwed by Singal and Ginder ‘DNA Methylation’ Blood, Vol. 93 No. 12 (Jun. 15, 1999): pp. 4059-4070. Further examples of methylation linked oncogenesis include:

Head and neck cancer (Sanchez-Cespedes M et al., “Gene promoter hypemethylation in tumours and serum of head and neck cancer patients” Cancer Res. Feb. 15, 2000;60 (4):892-5) Hodgkin's disease (Garcia J F et al “Loss of p16 protein expression associated with methylation of the p16INK4A gene is a frequent finding in Hodgkin's disease” Lab invest December 1999; 79 (12):1453-9):

Gastric cancer (Yanagisawa Y et al. “Methylation of the hMLH1 promoter in familial gastric cancer with microsatellite instability” Int J Cancer Jan. 1, 2000; 85 (1):50-3):

The identification of the methylation dependant regulation of cancer genes has opened up the possibility of creating alternative methods of cancer treatment and diagnosis. Treatment with DNA methylation inhibitors has been shown to restore gene expression of the key tumor suppressor genes and oncogenes gene p16, Bender et. al. “Inhibition of DNA methylation by 5-aza-2′-deoxycytidine suppresses the growth of human tumor cell lines.” Cancer research 58; 95-101 (1998). This resulted in heritable levels of gene expression leading to suppression of growth in tumor cell lines.

Methylation based therapies could have considerable advantages over current methods of treatment, such as chemotherapy, surgery and radiotherapy. They may even provide a means of treating tumors which are resistant to conventional methods of therapy, as demonstrated by Soengas et al “Inactivation of the apoptosis effector Apaf-1 in malignant melanoma” Nature 409; 207-211(2001). In addition to the development of methylation specific therapies, experiments with Min mice have shown that inhibition of DNA methylation can suppress tumor initiation, Laird et. al. ‘Suppression of intestinal neoplasia by DNA hypomethylation’ Cell 81; 197-205 (1995). Furthermore, DNA methylation analysis may provide novel means for cancer diagnosis.

5-methylcytosine is the most frequent covalent base modification in the DNA of eukaryotic cells. It plays a role, for example, in the regulation of the transcription, in genetic imprinting. and in tumorigenesis. Therefore, the identification of 5-methylcytosine as a component of genetic information is of considerable interest. However, 5-methylcytosine positions cannot be identified by sequencing since 5-methylcytosine has the same base pairing behavior as cytosine. Moreover, the epigenetic information carried by 5-methylcytosine is completely lost during PCR amplification.

A relatively new and currently the most frequently used method for analyzing DNA for 5-methylcytosine is based upon the specific reaction of bisulfite with cytosine which, upon subsequent alkaline hydrolysis, is converted to uracil which corresponds to thymidine in its base pairing behavior. However, 5-methylcytosine remains unmodified under these conditions.

Consequently, the original DNA is converted in such a manner that methylcytosine, which originally could not be distinguished from cytosine by its hybridization behavior, can now be detected as the only remaining cytosine using “normal” molecular biological techniques, for example, by amplification and hybridization or sequencing. All of these techniques are based on base pairing which can now be fully exploited. In terms of sensitivity, the prior art is defined by a method which encloses the DNA to be analyzed in an agarose matrix, thus preventing the diffusion and renaturation of the DNA (bisulfite only reacts with single-stranded DNA), and which replaces all precipitation and purification steps with fast dialysis (Olek A, Oswald J, Walter J. A modified and improved method for bisulphite based cytosine methylation analysis. Nucleic Acids Res. Dec. 15, 1996;24(24):5064-6). Using this method, it is possible to analyze individual cells, which illustrates the potential of the method. However, currently only individual regions of a length of up to approximately 3000 base pairs are analyzed, a global analysis of cells for thousands of possible methylation events is not possible. However, this method cannot reliably analyze very small fragments from small sample quantities either. These are lost through the matrix in spite of the diffusion protection.

An overview of the further known methods of detecting 5-methylcytosine may be gathered from the following review article: Rein, T., DePamphilis, M. L., Zorbas, H., Nucleic Acids Res. 1998, 26, 2255.

To date, barring few exceptions (e.g., Zeschnigk M, Lich C, Buiting K, Doerfler W, Horsthemke B. A single-tube PCR test for the diagnosis of Angelman and Prader-Willi syndrome based on allelic methylation differences at the SNRPN locus. Eur J Hum Genet. March-April 1997;5(2):94-8) the bisulfite technique is only used in research. Always, however, short, specific fragments of a known gene are amplified subsequent to a bisulfite treatment and either completely sequenced (Olek A, Walter J. The pre-implantation ontogeny of the H19 methylation imprint. Nat Genet. November 1997;17(3):275-6) or individual cytosine positions are detected by a primer extension reaction (Gonzalgo M L, Jones P A. Rapid quantitation of methylation differences at specific sites using methylation-sensitive single nucleotide primer extension (Ms-SNuPE). Nucleic Acids Res. Jun. 15, 1997;25(12):2529-31, WO Patent 9500669) or by enzymatic digestion (Xiong Z, Laird P W. COBRA: a sensitive and quantitative DNA methylation assay. Nucleic Acids Res. Jun. 15, 1997;25(12):2532-4). In addition, detection by hybridization has also been described (Olek et al., WO 99 28498).

Further publications dealing with the use of the bisulfite technique for methylation detection in individual genes are: Grigg G, Clark S. Sequencing 5-methylcytosine residues in genomic DNA. Bioessays. June 1994;16(6):431-6, 431; Zeschnigk M, Schmitz B, Dittrich B, Buiting K, Horsthemke B, Doerfler W. Imprinted segments in the human genome: different DNA methylation patterns in the Prader-Willi/Angelman syndrome region as determined by the genomic sequencing method. Hum Mol Genet. March 1997;6(3):387-95; Feil R, Charlton J, Bird A P, Walter J, Reik W. Methylation analysis on individual chromosomes: improved protocol for bisulphite genomic sequencing. Nucleic Acids Res. Feb. 25, 1994;22(4):695-6; Martin V, Ribieras S, Song-Wang X, Rio M C, Dante R. Genomic sequencing indicates a correlation between DNA hypomethylation in the 5′ region of the pS2 gene and its expression in human breast cancer cell lines. Gene. May 19, 1995;157(1-2):261-4; WO 97/46705, WO 95/15373 and WO 98/45560.

An overview of the Prior Art in oligomer array manufacturing can be gathered from a special edition of Nature Genetics (Nature Genetics Supplement, Volume 21 January 1999), published in January 1999, and from the literature cited therein.

Fluorescently labeled probes are often used for the scanning of immobilized DNA arrays. The simple attachment of Cy3 and Cy5 dyes to the 5′-OH of the specific probe are particularly suitable for fluorescence labels. The detection of the fluorescence of the hybridized probes may be carried out, for example via a confocal microscope. Cy3 and Cy5 dyes, besides many others, are commercially available.

Matrix Assisted Laser Desorption Ionization Mass Spectrometry (MALDI-TOF) is a very efficient development for the analysis of biomolecules (Karas M, Hillenkamp F. Laser desorption ionization of proteins with molecular masses exceeding 10,000 daltons. Anal Chem. Oct. 15, 1988;60(20):2299-301). An analyte is embedded in a light-absorbing matrix. The matrix is evaporated by a short laser pulse thus transporting the analyte molecule into the vapor phase in an unfragmented manner. The analyte is ionized by collisions with matrix molecules. An applied voltage accelerates the ions into a field-free flight tube. Due to their different masses, the ions are accelerated at different rates. Smaller ions reach the detector sooner than bigger ones.

MALDI-TOF spectrometry is excellently suited to the analysis of peptides and proteins. The analysis of nucleic acids is somewhat more difficult (Gut I G, Beck S. DNA and Matrix Assisted Laser Desorption Ionization Mass Spectrometry. Current Innovations and Future Trends. 1995, 1; 147-57). The sensitivity to nucleic acids is approximately 100 times worse than to peptides and decreases disproportionally with increasing fragment size. For nucleic acids having a multiply negatively charged backbone, the ionization process via the matrix is considerably less efficient. In MALDI-TOF spectrometry, the selection of the matrix plays an eminently important role. For the desorption of peptides, several very efficient matrixes have been found which produce a very fine crystallization. There are now several responsive matrixes for DNA, however, the difference in sensitivity has not been reduced. The difference in sensitivity can be reduced by chemically modifying the DNA in such a manner that it becomes more similar to a peptide. Phosphorothioate nucleic acids in which the usual phosphates of the backbone are substituted with thiophosphates can be converted into a charge-neutral DNA using simple alkylation chemistry (Gut I G, Beck S. A procedure for selective DNA alkylation and detection by mass spectrometry. Nucleic Acids Res. Apr. 25, 1995;23(8):1367-73). The coupling of a charge tag to this modified DNA results in an increase in sensitivity to the same level as that found for peptides. A further advantage of charge tagging is the increased stability of the analysis against impurities which make the detection of unmodified substrates considerably more difficult.

Genomic DNA is obtained from DNA of cell, tissue or other test samples using standard methods. This standard methodology is found in references such as Fritsch and Maniatis eds., Molecular Cloning: A Laboratory Manual, 1989.

Problem Definition

The present invention is intended to provide oligonucleotides and/or PNA-oligomers for detecting cytosine methylations as well as a method which is particularly suitable for the diagnosis and/or therapy of genetic and epigenetic parameters of genes associated with tumor suppressor genes and oncogenes. The present invention is based on the discovery that, cytosine methylation patterns are particularly suitable for the diagnosis and/or therapy of diseases associated with tumor suppressor genes and oncogenes.

Description

The object of the present invention is to provide the chemically modified DNA of genes associated with tumor suppressor genes and oncogenes, as well as oligonucleotides and/or PNA-oligomers for detecting cytosine methylations, as well as a method which is particularly suitable for the diagnosis and/or therapy of genetic and epigenetic parameters of genes associated with tumor suppressor genes and oncogenes. The present invention is based on the discovery that genetic and epigenetic parameters and, in particular, the cytosine methylation pattern of genes associated with tumor suppressor genes and oncogenes are particularly suitable for the diagnosis and/or therapy of diseases associated with tumor suppressor genes and oncogenes.

This objective is achieved according to the present invention using a nucleic acid containing a sequence of at least 18 bases in length of the chemically pretreated DNA of genes associated with tumor suppressor genes and oncogenes according to one of Seq. ID No. 1 through Seq. ID No. 536 and sequences complementary thereto and/or a chemically pretreated DNA of genes associated with tumor suppressor genes and oncogenes according to sequences according to table 1. In the table, after the listed gene designations, the respective data bank numbers (accession numbers) are specified which define the appertaining gene sequences as unique. GenBank was used as the underlying data bank, which is located at the National Institute of Health at the internet address www.ncbi.nlm.nih.gov.

The chemically modified nucleic acid could heretofore not be connected with the ascertainment of genetic and epigenetic parameters.

The object of the present invention is further achieved by an oligonucleotide or oligomer for detecting the cytosine methylation state in chemically pretreated DNA, containing at least one base sequence having a length of at least 13 nucleotides which hybridizes to a chemically pretreated DNA of genes associated with tumor suppressor genes and oncogenes according to Seq. ID No. 1 through Seq. ID No. 536 and sequences complementary thereto and/or a chemically pretreated DNA of genes associated with tumor suppressor genes and oncogenes according to sequences according to table 1. The oligomer probes according to the present invention constitute important and effective tools which, for the first time, make it possible to ascertain the genetic and epigenetic parameters of genes associated with tumor suppressor genes and oncogenes. The base sequence of the oligomers preferably contains at least one CpG dinucleotide. The probes may also exist in the form of a PNA (peptide nucleic acid) which has particularly preferred pairing properties. Particularly preferred are oligonucleotides according to the present invention in which the cytosine of the CpG dinucleotide is the 5th-9th nucleotide from the 5′-end of the 13-mer; in the case of PNA-oligomers, it is preferred for the cytosine of the CpG dinucleotide to be the 4th-6th nucleotide from the 5′-end of the 9-mer.

The oligomers according to the present invention are normally used in so called “sets” which contain at least one oligomer for each of the CpG dinucleotides of the sequences of Seq. ID No. 1 through Seq. ID No. 536 and sequences complementary thereto and/or a chemically pretreated DNA of genes associated with tumor suppressor genes and oncogenes according to sequences according to table 1. Preferred is a set which contains at least one oligomer for each of the CpG dinucleotides from one of Seq. ID No. 1 through Seq. ID No. 536 and sequences complementary thereto and/or a chemically pretreated DNA of genes associated with tumor suppressor genes and oncogenes according to sequences according to table 1.

Moreover, the present invention makes available a set of at least two oligonucleotides which can be used as so-called “primer oligonucleotides” for amplifying DNA sequences of one of Seq. ID No. 1 through Seq. ID No. 536 and sequences complementary thereto and/or a chemically pretreated DNA of genes associated with tumor suppressor genes and oncogenes according to sequences according to table 1, or segments thereof.

In the case of the sets of oligonucleotides according to the present invention, it is preferred that at least one oligonucleotide is bound to a solid phase.

The present invention moreover relates to a set of at least 10 n (oligonucleotides and/or PNA-oligomers) used for detecting the cytosine methylation state in chemically pretreated genomic DNA (Seq. ID No. 1 through Seq. ID No. 536 and sequences complementary thereto and/or a chemically pretreated DNA of genes associated with tumor suppressor genes and oncogenes according to sequences according to table 1). These probes enable diagnosis and/or therapy of genetic and epigenetic parameters of genes associated with tumor suppressor genes and oncogenes. The set of oligomers may also be used for detecting single nucleotide polymorphisms (SNPS) in the chemically pretreated DNA of genes associated with tumor suppressor genes and oncogenes according to one of Seq. ID No. 1 through Seq. ID No. 536 and sequences complementary thereto and/or a chemically pretreated DNA of genes associated with tumor suppressor genes and oncogenes according to sequences according to table 1.

According to the present invention, it is preferred that an arrangement of different oligonucleotides and/or PNA-oligomers (a so-called “array”) made available by the present invention is present in a manner that it is likewise bound to a solid phase. This array of different oligonucleotide- and/or PNA-oligomer sequences can be characterized in that it is arranged on the solid phase in the form of a rectangular or hexagonal lattice. The solid phase surface is preferably composed of silicon, glass, polystyrene, aluminum, steel, iron, copper, nickel, silver, or gold. However, nitrocellulose as well as plastics such as nylon which can exist in the form of pellets or also as resin matrices are possible as well.

Therefore, a further subject matter of the present invention is a method for manufacturing an array fixed to a carrier material for analysis in connection with diseases associated with tumor suppressor genes and oncogenes in which method at least one oligomer according to the present invention is coupled to a solid phase. Methods for manufacturing such arrays are known, for example, from U.S. Pat. No. 5,744,305 by means of solid-phase chemistry and photolabile protecting groups.

A further subject matter of the present invention relates to a DNA chip for the analysis of diseases associated with tumor suppressor genes and oncogenes which contains at least one nucleic acid according to the present invention. DNA chips are known, for example, from U.S. Pat. No. 5,837,832.

Moreover, a subject matter of the present invention is a kit which may be composed, for example, of a bisulfite-containing reagent, a set of primer oligonucleotides containing at least two oligonucleotides whose sequences in each case correspond or are complementary to an 18 base long segment of the base sequences specified in the appendix (Seq. ID No. 1 through Seq. ID No. 536 and sequences complementary thereto and/or a chemically pretreated DNA of genes associated with tumor suppressor genes and oncogenes according to sequences according to table 1), oligonucleotides and/or PNA-oligomers as well as instructions for carrying out and evaluating the described method. However, a kit along the lines of the present invention can also contain only part of the aforementioned components.

The present invention also makes available a method for ascertaining genetic and/or epigenetic parameters of genes associated with the cycle cell by analyzing cytosine methylations and single nucleotide polymorphisms, including the following steps:

In the first step of the method, a genomic DNA sample is chemically treated in such a manner that cytosine bases which are unmethylated at the 5′-position are converted to uracil, thymine, or another base which is dissimilar to cytosine in terms of hybridization behavior. This will be understood as ‘chemical pretreatment’ hereinafter.

The genomic DNA to be analyzed is preferably obtained form usual sources of DNA such as cells or cell components, for example, cell lines, biopsies, blood, sputum, stool, urine, cerebral-spinal fluid, tissue embedded in paraffin such as tissue from eyes, intestine, kidney, brain, heart, prostate, lung, breast or liver, histologic object slides, or combinations thereof.

The above described treatment of genomic DNA is preferably carried out with bisulfite (hydrogen sulfite, disulfite) and subsequent alkaline hydrolysis which results in a conversion of non-methylated cytosine nucleobases to uracil or to another base which is dissimilar to cytosine in terms of base pairing behavior.

Fragments of the chemically pretreated DNA are amplified, using sets of primer oligonucleotides according to the present invention, and a, preferably heat-stable polymerase. Because of statistical and practical considerations, preferably more than ten different fragments having a length of 100-2000 base pairs are amplified. The amplification of several DNA segments can be carried out simultaneously in one and the same reaction vessel. Usually, the amplification is carried out by means of a polymerase chain reaction (PCR).

In a preferred embodiment of the method, the set of primer oligonucleotides includes at least two olignonucleotides whose sequences are each reverse complementary or identical to an at least 18 base-pair long segment of the base sequences specified in the appendix (Seq. ID No. 1 through Seq. ID No. 536 and sequences complementary thereto and/or a chemically pretreated DNA of genes associated with tumor suppressor genes and oncogenes according to sequences according to table 1). The primer oligonucleotides are preferably characterized in that they do not contain any CpG dinucleotides.

According to the present invention, it is preferred that at least one primer oligonucleotide is bonded to a solid phase during amplification. The different oligonucleotide and/or PNA-oligomer sequences can be arranged on a plane solid phase in the form of a rectangular or hexagonal lattice, the solid phase surface preferably being composed of silicon, glass, polystyrene, aluminum, steel, iron, copper, nickel, silver, or gold, it being possible for other materials such as nitrocellulose or plastics to be used as well.

The fragments obtained by means of the amplification can carry a directly or indirectly detectable label. Preferred are labels in the form of fluorescence labels, radionuclides, or detachable molecule fragments having a typical mass which can be detected in a mass spectrometer, it being preferred that the fragments that are produced have a single positive or negative net charge for better detectability in the mass spectrometer. The detection may be carried out and visualized by means of matrix assisted laser desorption/ionization mass spectrometry (MALDI) or using electron spray mass spectrometry (ESI).

The amplificates obtained in the second step of the method are subsequently hybridized to an array or a set of oligonucleotides and/or PNA probes. In this context, the hybridization takes place in the manner described in the following. The set of probes used during the hybridization is preferably composed of at least 10 oligonucleotides or PNA-oligomers. In the process, the amplificates serve as probes which hybridize to oligonucleotides previously bonded to a solid phase. The non-hybridized fragments are subsequently removed. Said oligonucleotides contain at least one base sequence having a length of 13 nucleotides which is reverse complementary or identical to a segment of the base sequences specified in the appendix, the segment containing at least one CpG dinucleotide. The cytosine of the CpG dinucleotide is the 5th to 9th nucleotide from the 5′-end of the 13-mer. One oligonucleotide exists for each CpG dinucleotide. Said PNA-oligomers contain at least one base sequence having a length of 9 nucleotides which is reverse complementary or identical to a segment of the base sequences specified in the appendix, the segment containing at least one CpG dinucleotide. The cytosine of the CpG dinucleotide is the 4th to 6th nucleotide seen from the 5′-end of the 9-mer. One oligonucleotide exists for each CpG dinucleotide.

In the fourth step of the method, the non-hybridized amplificates are removed.

In the final step of the method, the hybridized amplificates are detected. In this context, it is preferred that labels attached to the amplificates are identifiable at each position of the solid phase at which an oligonucleotide sequence is located.

According to the present invention, it is preferred that the labels of the amplificates are fluorescence labels, radionuclides, or detachable molecule fragments having a typical mass which can be detected in a mass spectrometer. The mass spectrometer is preferred for the detection of the amplificates, fragments of the amplificates or of probes which are complementary to the amplificates, it being possible for the detection to be carried out and visualized by means of matrix assisted laser desorption/ionization mass spectrometry (MALDI) or using electron spray mass spectrometry (ESI).

The produced fragments may have a single positive or negative net charge for better detectability in the mass spectrometer. The aforementioned method is preferably used for ascertaining genetic and/or epigenetic parameters of genes associated with tumor suppressor genes and oncogenes.

The oligomers according to the present invention or arrays thereof as well as a kit according to the present invention are intended to be used for the diagnosis and/or therapy of diseases associated with tumor suppressor genes and oncogenes by analyzing methylation patterns of genes associated with tumor suppressor genes and oncogenes. According to the present invention, the method is preferably used for the diagnosis and/or therapy of important genetic and/or epigenetic parameters within genes associated with tumor suppressor genes and oncogenes.

The method according to the present invention is used, for example, for the diagnosis and/or therapy of solid tumors and cancers

The nucleic acids according to the present invention of Seq. ID No. 1 through Seq. ID No. 536 and sequences complementary thereto and/or a chemically pretreated DNA of genes associated with tumor suppressor genes and oncogenes according to sequences according to table 1 can be used for the diagnosis and/or therapy of genetic and/or epigenetic parameters of genes associated with tumor suppressor genes and oncogenes.

The present invention moreover relates to a method for manufacturing a diagnostic agent and/or therapeutic agent for the diagnosis and/or therapy of diseases associated with tumor suppressor genes and oncogenes by analyzing methylation patterns of genes associated with tumor suppressor genes and oncogenes, the diagnostic agent and/or therapeutic agent being characterized in that at least one nucleic acid according to the present invention is used for manufacturing it, possibly together with suitable additives and auxiliary agents.

A further subject matter of the present invention relates to a diagnostic agent and/or therapeutic agent for diseases associated with tumor suppressor genes and oncogenes by analyzing methylation patterns of genes associated with tumor suppressor genes and oncogenes, the diagnostic agent and/or therapeutic agent containing at least one nucleic acid according to the present invention, possibly together with suitable additives and auxiliary agents.

The present invention moreover relates to the diagnosis and/or prognosis of events which are disadvantageous to patients or individuals in which important genetic and/or epigenetic parameters within genes associated with tumor suppressor genes and oncogenes said parameters obtained by means of the present invention may be compared to another set of genetic and/or epigenetic parameters, the differences serving as the basis for a diagnosis and/or prognosis of events which are disadvantageous to patients or individuals.

In the context of the present invention the term “hybridization” is to be understood as a bond of an oligonucleotide to a completely complementary sequence along the lines of the Watson-Crick base pairings in the sample DNA, forming a duplex structure. To be understood by “stringent hybridization conditions” are those conditions in which a hybridization is carried out at 60° C. in 2.5×SSC buffer, followed by several washing steps at 37° C. in a low buffer concentration, and remains stable.

The term “functional variants” denotes all DNA sequences which are complementary to a DNA sequence, and which hybridize to the reference sequence under stringent conditions and have an activity similar to the corresponding polypeptide according to the present invention.

In the context of the present invention, “genetic parameters” are mutations and polymorphisms of genes associated with tumor suppressor genes and oncogenes and sequences further required for their regulation. To be designated as mutations are, in particular, insertions, deletions, point mutations, inversions and polymorphisms and, particularly preferred, SNPs (single nucleotide polymorphisms).

In the context of the present invention, “epigenetic parameters” are, in particular, cytosine methylations and further chemical modifications of DNA bases of genes associated with tumor suppressor genes and oncogenes and sequences further required for their regulation. Further epigenetic parameters include, for example, the acetylation of histones which, however, cannot be directly analyzed using the described method but which, in turn, correlates with the DNA methylation.

In the following, the present invention will be explained in greater detail on the basis of the sequences and examples with reference to the accompanying figure without being limited thereto.

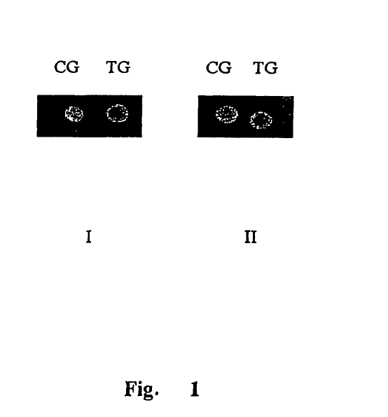

### FIG. 1

FIG. 1 shows the hybridisation of fluorescent labelled amplificates to a surface bound olignonucleotide. Sample I being from pilocytic astrocytoma (brain tumor) tissue and sample II being from astrocytoma grade II (brain tumor) tissue. Flourescence at a spot shows hybridisation of the amplificate to the olignonucleotide. Hybridisation to a CG olignonucleotide denotes methylation at the cytosine position being analysed, hybridisation to a TG olignonucleotide denotes no methylation at the cytosine position being analysed.

### Sequence ID Nos 1 to 536

Sequence ID Nos 1 to 536 show sequences of the chemically pretreated DNA of genes associated with tumor suppressor genes and oncogenes according to the invention. Sequences having odd sequence numbers (e.g., Seq. ID No. 1, 3, 5, . . . ) exhibit in each case sequences of the chemically pretreated genomic DNAs of different genes associated with tumor suppressor genes and oncogenes. Sequences having even sequence numbers (e.g., Seq. ID No. 2, 4, 6, . . . ) exhibit in each case the sequences of the chemically pretreated genomic DNAs of genes associated with tumor suppressor genes and oncogenes which are complementary to the preceeding sequences (e.g., the complementary sequence to Seq. ID No. 1 is Seq. ID No. 2, the complementary sequence to Seq. ID No. 3 is Seq. ID No. 4, etc.).

### Sequence ID Nos 537 to 540

Sequence ID Nos 537 to 540 show the sequences of oligonucleotides used in Example 1

The following example relates to a fragment of a gene associated with tumor suppressor genes and oncogenes, in this case, MYC in which a specific CG-position is analyzed for its methylation status.

### EXAMPLE 1

Methylation Analysis in the Gene MYC Associated with Tumor Suppressor Genes and Oncogenes.

The following example relates to a fragment of the gene MYC in which a specific CG-position is to be analyzed for methylation.

In the first step, a genomic sequence is treated using bisulfite (hydrogen sulfite, disulfite) in such a manner that all cytosines which are not methylated at the 5-position of the base are modified in such a manner that a different base is substituted with regard to the base pairing behavior while the cytosines methylated at the 5-position remain unchanged.

If bisulfite solution is used for the reaction, then an addition takes place at the non-methylated cytosine bases. Moreover, a denaturating reagent or solvent as well as a radical interceptor must be present. A subsequent alkaline hydrolysis then gives rise to the conversion of non-methylated cytosine nucleobases to uracil. The chemically converted DNA (sequence ID 531) is then used for the detection of methylated cytosines. In the second method step, the treated DNA sample is diluted with water or an aqueous solution. Preferably, the DNA is subsequently desulfonated (10-30 min, 90-100° C.) at an alkaline pH value. In the third step of the method, the DNA sample is amplified in a polymerase chain reaction, preferably using a heat-resistant DNA polymerase. In the present case, cytosines of the gene MYC are analyzed. To this end, a defined fragment having a length of 831 bp is amplified with the specific primer oligonucleotides TTTTGTGTGGAGGGTAGTTG (sequence ID 537) and CCCCAAATAAACAAAATAACC (sequence ID 538). This amplificate serves as a sample which hybridizes to an oligonucleotide previously bonded to a solid phase, forming a duplex structure, for example TAAGGATGCGGTTTGTTA (sequence ID 539), the cytosine to be detected being located at position 60 of the amplificate. The detection of the hybridization product is based on Cy3 and Cy5 flourescently labeled primer oligonucleotides which have been used for the amplification. A hybridization reaction of the amplified DNA with the oligonucleotide takes place only if a methylated cytosine was present at this location in the bi sulfite-treated DNA. Thus, the methylation status of the specific cytosine to be analyzed is inferred from the hybridization product.

In order to verify the methylation status of the position, a sample of the amplificate is further hybridized to another oligonucleotide previously bonded to a solid phase. Said olignonucleotide is identical to the oligonucleotide previously used to analyze the methylation status of the sample, with the exception of the position in question. At the position to be analysed said oligonucleotide comprises a thymine base as opposed to a cytosine base i.e. sequence TAAGGATGTGGTTTGTTA (sequence ID 540). Therefore, the hybridisation reaction only takes place if an unmethylated cytosine was present at the position to be analysed.

The analysis was carried out on two tissue samples, Sample 1 from a pilocytic astrocytoma (brain tumor), and Sample 2 from an astrocytoma grade II (brain tumor). From the results (FIG. 1) it can be seen that Sample 1 was unmethylated at position 60 of the amplificate whereas Sample 2 contained a mixture of both methylated and unmethylated cells at the same position.

### EXAMPLE 2

Diagnosis of Diseases Associated with Tumor Suppressor Genes and Oncogenes

In order to relate the methylation patterns to one of the diseases associated with tumor suppressor genes and oncogenes, it is initially required to analyze the DNA methylation patterns of a group of diseased and of a group of healthy patients. These analyses are carried out, for example, analogously to example 1. The results obtained in this manner are stored in a data base and the CpG dinucleotides which are methylated differently between the two groups are identified. This can be carried out by determining individual CpG methylation rates as can be done, for example, in a relatively imprecise manner, by sequencing or else, in a very precise manner, by a methylation-sensitive “primer extension reaction”. It is also possible for the entire methylation status to be analyzed simultaneously, and for the patterns to be compared, for example, by clustering analyses which can be carried out, for example, by a computer.

Subsequently, it is possible to allocate the examined patients to a specific therapy group and to treat these patients selectively with an individualized therapy.

Example 2 can be carried out, for example, for the following diseases: Solid tumors and cancers.

TABLE 1
Listing of preferred genes associated with tumor suppressor genes and
oncogenes according to the invention.

| | Database entry No. (GenBank, internet address |
|---|---|
| Gene | www.ncbi.nlm.nih.gov) |
| BRCA2 | (U43746), |
| E2F1 | (M96577), |
| ELE1 | (X71413), |
| MN/CA9 | (Z54349), |
| PVT1 | (M26714), |
| SAC2 | (AK001725), |
| TEM8 | (AK025429), |
| TM4SF1 | (X01394), |
| TNFSF11 | (AF053712), |
| AXL | (NM_021913 & NM_001699), |
| CCND3 | (NM_001760), |
| CSF1R | (NM_005211), |
| MGMT | (NM_002412), |
| NOV | (NM_002514), |
| SFRS8 | (NM_004592), |
| TNFRSF6 | (NM_000043), |
| TPD52 | (NM_005079), |
| AKT1 | (NM_005163), |
| BCL2 | (NM_000633 & NM_000657), |
| CBL | (NM_005188), |
| CBLC | (NM_012116), |
| CRK | (NM_005206 & NM_016823), |
| DCC | (NM_005215), |
| EPHA1 | (NM_005232), |
| EPHA3 | (NM_005233), |
| ETS1 | (NM_005238), |
| ETV5 | (NM_004454), |
| ETV6 | (NM_001987), |
| FGF3 | (NM_005247), |
| FGF4 | (NM_002007), |
| FHIT | (NM_002012), |
| GLTSCR1 | (NM_015711), |
| GPS1 | (NM_004127), |
| GROS1 | (NM_022356), |
| HIC1 | (NM_006497), |
| IGFBP7 | (NM_001553), |
| KISS1 | (NM_002256), |
| KRAS2 | (NM_004985), |
| LATS1 | (NM_004690), |
| LOC51213 | (NM_016383), |
| MUC1 | (NM_002456), |
| MUC2 | (NM_002457), |
| N33 | (NM_006765), |
| PTTG1IP | (NM_004339), |
| SE20-4 | (NM_022117), |
| SE70-2 | (NM_022118), |
| SFN | (NM_006142), |
| ST7 | (NM_013437), |
| SUPT3H | (NM_003599), |
| SUPT6H | (NM_003170), |
| TEM1 | (NM_020404), |
| TERT | (NM_003219), |
| THRB | NM_000461), |
| TIMP2 | (NM_003255), |
| TMEFF1 | (NM_003692), |
| TNFAIP6 | (NM_007115), |
| TNFRSF10A | (NM_003844), |
| TNFRSF10B | (NM_003842), |
| TNFRSF10C | (NM_003841), |
| TNFRSF11A | (NM_003840), |
| TNFRSF1A | (NM_001065), |
| TNFSF12 | (NM_003809), |
| TNFSF13 | (NM_003808), |
| TNFSF15 | (NM_005118), |
| TNFSF18 | (NM_005092), |
| TP63 | (NM_003722), |
| TSSC1 | (NM_003310), |
| VDR | (NM_000376), |
| YES1 | (NM_005433), |
| FOXG1A | (NM_004471), |
| GRF2 | (NM_005312), |
| HSPC070 | (NM_014160), |
| RAB3A | (NM_002866), |
| RAB5A | (NM_004162), |
| APC | (NM_000038), |
| BMI1 | (NM_005180), |
| CHES1 | (NM_005197), |
| SMT3H1 | (NM_006936), |
| TIAM1 | (NM_003253), |
| VAV1 | (NM_005428), |
| MCF2 | (NM_005369), |
| MSH2 | (NM_000251), |
| ERBB4 | (NM_005235), |
| FOXG1B | (NM_005249), |
| TACSTD1 | (NM_002354), |
| TRA1 | (NM_003299), |
| FOXG1B | (NM_005249), |
| TACSTD1 | (NM_002354), |
| FLI1 | (NM_002017) |

1. A nucleic acid comprising a sequence at least 18 bases in length of a segment of the chemically pretreated DNA of genes associated with tumor suppressor genes and oncogenes according to one of the sequences taken from the group of Seq. ID No. 1 to Seq. ID No. 536 and sequences complementary thereto.

2. A nucleic acid comprising a sequence at least 18 bases in length of a segment of the chemically pretreated DNA of genes associated with tumor suppressor genes and oncogenes according to one of the sequences according to one of the genes BRCA2 (U43746), E2F1 (M96577), ELE1 (X71413), MN/CA9 (Z54349), PVT1 (M26714), SAC2 (AK001725), TEM8 (AK025429), TM4SF1 (X01394), TNFSF11 (AF053712), AXL (NM_{—}021913&NM_{—}001699), CCND3 (NM_{—}001760), CSF1R (NM_{—}005211), MGMT (NM_{—}002412), NOV (NM_{—}002514), SFRS8 (NM_{—}004592), TNFRSF6 (NM_{—}000043), TPD52 (NM_{—}005079), AKT1 (NM_{—}005163), BCL2 (NM_{—}000633&NM_{—}000657), CBL (NM_{—}005188), CBLC (NM_{—}012116), CRK (NM_{—}005206&NM_{—}016823), DCC (NM_{—}005215), EPHA1 (NM_{—}005232), EPHA3 (NM_{—}005233), ETS1 (NM_{—}005238), ETV5 (NM_{—}004454), ETV6 (NM_{—}001987), FGF3 (NM_{—}005247), FGF4 (NM_{—}002007), FHIT (NM_{—}002012), GLTSCR1 (NM_{—}015711), GPS1 (NM_{—}004127), GROS1(NM_{—}022356), HIC1 (NM_{—}006497), IGFBP7 (NM_{—}001553), KISS1 (NM_{—}002256), KRAS2 (NM_{—}004985), LATS1 (NM_{—}004690), LOC51213 (NM_{—}016383), MUC1 (NM_{—}002456), MUC2 (NM_{—}002457), N33 (NM_{—}006765), PTTG1IP (NM_{—}004339), SE20-4 (NM_{—}022117), SE70-2 (NM_{—}022118), SFN (NM_{—}006142), ST7 (NM_{—}013437), SUPT3H (NM_{—}003599), SUPT6H (NM_{—}003170), TEM1 (NM_{—}020404), TERT (NM_{—}003219), THRB NM_{—}000461), TIMP2 (NM_{—}003255), TMEFF1 (NM_{—}003692), TNFAIP6 (NM_{—}007115), TNFRSF10A (NM_{—}003844), TNFRSF10B (NM_{—}003842), TNFRSF10C (NM_{—}003841), TNFRSF11A (NM_{—}003840), TNFRSF1A (NM_{—}001065), TNFSF12 (NM_{—}003809), TNFSF13 (NM_{—}003808), TNFSF15 (NM_{—}005118), TNFSF18 (NM_{—}005092), TP63 (NM_{—}003722), TSSC1 (NM_{—}003310), VDR (NM_{—}000376), YES1 (NM_{—}005433), FOXG1A (NM_{—}004471), GRF2 (NM_{—}005312), HSPC070 (NM_{—}014160), RAB3A (NM_{—}002866), RAB5A (NM_{—}004162), APC (NM_{—}000038), BMI1 (NM_{—}005180), CHES1 (NM_{—}005197), SMT3H1 (NM_{—}006936), TIAM1 (NM_{—}003253), VAV1 (NM_{—}005428), MCF2 (NM_{—}005369), MSH2 (NM_{—}000251), ERBB4 (NM_{—}005235), FOXG1B (NM_{—}005249), TACSTD1 (NM_{—}002354), TRA1 (NM_{—}003299), FOXG1B (NM_{—}005249), TACSTD1 (NM_{—}002354), FLI1 (NM_{—}002017) and sequences complementary thereto.

3. An oligomer, in particular an oligonucleotide or peptide nucleic acid (PNA)-oligomer, said oligomer comprising in each case at least one base sequence having a length of at least 9 nucleotides which hybridizes to or is identical to a chemically pretreated DNA of genes associated with the cell cycle according to one of the Seq. ID No. 1 through Seq. ID No. 536 and sequences complementary thereto.

4. The oligomer as recited in claim 3; wherein the base sequence comprises at least one CpG dinucleotide.

5. The oligomer as recited in claim 4; characterized in that the cytosine of the CpG dinucleotide is located approximately in the middle third of the oligomer.

6. A set of oligomers, comprising at least two oligomers according to one of claims 3 to 5.

7. A set of oligomers as recited in claim 6, comprising oligomers for detecting the methylation state of all CpG dinucleotides from one of the sequences of Seq. ID 1 through Seq. ID 536 or to a chemically pretreated DNA of a gene according to claim 2, and sequences complementary thereto.

8. A set of at least two oligonucleotides as recited in claim 3 which can be used as primer oligonucleotides for the amplification of DNA sequences of one of Seq. ID 1 through Seq. ID 536 and sequences complementary thereto and/or to a chemically pretreated DNA of a gene according to claim 2, and sequences complementary thereto, or segments thereof.

9. A set of oligonucleotides as recited in claim 8, characterized in that at least one oligonucleotide is bound to a solid phase.

10. A set of oligomer probes for detecting the cytosine methylation state and/or single nucleotide polymorphisms (SNPs) in chemically pretreated genomic DNA according to claim 1 and/or a chemically pretreated DNA of a gene according to claim 2.

11. A method for manufacturing an arrangement of different oligomers (array) fixed to a carrier material for analyzing diseases associated with the methylation state of the CpG dinucleotides of one of the Seq. ID 1 through Seq. ID 536 and sequences complementary thereto and/or a chemically pretreated DNA of a gene according to claim 2,
wherein at least one oligomer according to one of the claims 3 through 5 is coupled to a solid phase.

12. An arrangement of different oligomers (array), obtainable according to claim 11.

13. An array of different oligonucleotide- and/or PNA-oligomer sequences as recited in claim 12, characterized in that these are arranged on a plane solid phase in the form of a rectangular or hexagonal lattice.

14. The array as recited in one of the claims 12 or 13, characterized in that the solid phase surface is composed of silicon, glass, polystyrene, aluminum, steel, iron, copper, nickel, silver, or gold.

15. A DNA- and/or PNA-array for analyzing diseases associated with the methylation state of genes, comprising at least one nucleic acid according to one of the preceeding claims.

16. A method for ascertaining genetic and/or epigenetic parameters for the diagnosis and/or therapy of existing diseases or the predisposition to specific diseases by analyzing cytosine methylations, characterized in that the following steps are carried out:
a) in a genomic DNA sample, cytosine bases which are unmethylated at the 5-position are converted, by chemical treatment, to uracil or another base which is dissimilar to cytosine in terms of hybridization behavior;
b) fragments of the chemically pretreated genomic DNA are amplified using sets of primer oligonucleotides according to claim 8 or 9 and a polymerase, the amplificates carrying a detectable label;
c) Amplificates are hybridized to a set of oligonucleotides and/or PNA probes according to the claims 6 or 7, or else to an array according to one of the claims 12 through 15;
d) the hybridized amplificates are subsequently detected.

17. The method as recited in claim 16, characterized in that the chemical treatment is carried out by means of a solution of a bisulfite, hydrogen sulfite or disulfite.

18. The method as recited in one of the claims 16 or 17, characterized in that more than ten different fragments having a length of 100-2000 base pairs are amplified.

19. The method as recited in one of the claims 16 through 18, characterized in that the amplification of several DNA segments is carried out in one reaction vessel.

20. The method as recited in one of the claims 16 through 19, characterized in that the polymerase is a heat-resistant DNA polymerase.

21. The method as recited in claim 20, characterized in that the amplification is carried out by means of the polymerase chain reaction (PCR).

22. The method as recited in one of the claims 16 through 21, characterized in that the labels of the amplificates are fluorescence labels.

23. The method as recited in one of the claims 16 through 21, characterized in that the labels of the amplificates are radionuclides.

24. The method as recited in one of the claims 16 through 21, characterized in that the labels of the amplificates are detachable molecule fragments having a typical mass which are detected in a mass spectrometer.

25. The method as recited in one of the claims 16 through 21, characterized in that the amplificates or fragments of the amplificates are detected in the mass spectrometer.

26. The method as recited in any of the claims 24 or 25, characterized in that the produced fragments have a single positive or negative net charge for better detectability in the mass spectrometer

27. The method as recited in one of the claims 24 through 26, characterized in that detection is carried out and visualized by means of matrix assisted laser desorption/ionization mass spectrometry (MALDI) or using electron spray mass spectrometry (ESI).

28. The method as recited in one of the claims 16 through 27, characterized in that the genomic DNA is obtained from cells or cellular components which contain DNA, sources of DNA comprising, for example, cell lines, biopsies, blood, sputum, stool, urine, cerebral-spinal fluid, tissue embedded in paraffin such as tissue from eyes, intestine, kidney, brain, heart, prostate, lung, breast or liver, histologic object slides, and all possible combinations thereof.

29. A kit comprising a bisulfite (=disulfite, hydrogen sulfite) reagent as well as oligonucleotides and/or PNA-oligomers according to one of the claims 3 through 5.

30. The use of a nucleic acid according to claim 1, of an oligonucleotide or PNA-oligomer according to one of the claims 3 through 5, of a kit according to claim 29, of an array according to one of the claims 12 through 15, of a set of oligonucleotides according to one of claims 6 through 9 for the diagnosis of solid tumors and cancers

31. The use of a nucleic acid according to claim 1, of an oligonucleotide or PNA-oligomer according to one of the claims 3 through 5, of a kit according to claim 29, of an array according to one of the claims 12 through 15, of a set of oligonucleotides according to one of claims 6 through 9 for the therapy of solid tumors and cancers

32. A kit, comprising a bisulfite (=disulfite, hydrogen sulfite) reagent as well as oligonucleotides and/or PNA-oligomers according to one of claims 3 through 5.
